(19) United States
(12) Patent Application Publication
(10) Pub. No.: US 2024/0248072 A1
(43) Pub. Date: Jul. 25, 2024

(54) DEVICE FOR ESTIMATING ACTIVE CLAY CONTENT, DEVICE FOR GENERATING LEARNED MODEL, METHOD FOR ESTIMATING ACTIVE CLAY CONTENT, AND METHOD FOR GENERATING LEARNED MODEL

(71) Applicant: SINTOKOGIO, LTD., Nagoya-shi, Aichi (JP)

(72) Inventors: Yohei NOGUCHI, Nagoya-shi (JP); Yuichi OGURA, Nagoya-shi (JP); Yoshinari NAKANO, Nagoya-shi (JP)

(21) Appl. No.: 18/561,388

(22) PCT Filed: Sep. 28, 2022
(86) PCT No.: PCT/JP2022/036118

§ 371 Date: Nov. 16, 2023

(30) Foreign Application Priority Data
Sep. 28, 2021 (JP) 2021-158416

(51) Int. Cl. G01N 33/24; G01N 3/00

(52) U.S. Cl. CPC G01N 33/24; G01N 3/00

(57) ABSTRACT

This invention estimates an active clay content in mixed sand. A processor (51) of an information processing device (50) executes: an obtaining step of obtaining information concerning mixed sand with which green sand containing clay is mixed; and an estimating step of estimating an active clay content in the mixed sand in accordance with the obtained information concerning the mixed sand.

### TECHNICAL FIELD

The present invention relates to a technique for estimating an active clay content.

### BACKGROUND ART

Molding sand used for molding is generated by mixing, with a sand mixer, a given amount of silica sand, a given amount of clay such as bentonite, and a given amount of an additive such as sea coal or starch. The mold is shaken out after used in casting. To green sand obtained as a result of the shake-out, clay and an additive are added again. A resultant is reused as molding sand. In this process, an amount of clay required to be added varies depending on the active clay content in the green sand. In order to properly reuse the green sand, it is necessary to measure the active clay content in the green sand.

There has been known a technique for measuring an active clay content in green sand. For example, Patent Literature 1 discloses a method for measuring an active clay content in green sand, the method including measuring a light absorbance or a light transmittance with use of (i) light having a wavelength with which a coloring agent exhibits a substantial peak light absorbance or a substantial peak light transmittance and (ii) at least one kind of light having a wavelength with which the coloring agent exhibits a stable light absorbance or a stable light transmittance. Patent Literature 2 discloses a device for measuring an active clay content, the device being configured to feed a reagent to a beaker-for-dispersing containing green sand charged thereto, to disperse an active clay component of the green sand in the reagent in the beaker-for-dispersing, to extract and dilute a dispersion liquid obtained by the reagent, and to analyze the diluted dispersion liquid by absorptiometric analysis. Patent Literature 3 discloses a technique for carrying out, by a washing method, fractional determination of an active component Na-Bt contained in active clay in green sand in accordance with a difference in swelling property and suspensibility in a dispersion liquid between Na-Bt and Ca-Bt.

### CITATION LIST

### Patent Literature

Patent Literature 1
Japanese Patent No. 3126652
Patent Literature 2
Japanese Patent Application Publication, Tokukaihei, No. 9-311131 (1997)
Patent Literature 3
Japanese Patent Application Publication, Tokukaihei, No. 11-290986 (1999)

### SUMMARY OF INVENTION

### Technical Problem

However, the measurement of the active clay content in accordance with the above-described conventional techniques needs time. Thus, the measurement of the active clay content is a rate-limiting factor, and decreases the rate of the entire sand treatment cycle.

An aspect of the present invention has an object to provide a technique for estimating an active clay content in mixed sand.

### Solution to Problem

In order to attain the above-described object, an active clay content estimation device in accordance with an aspect of the present invention includes one or more processors configured to execute an obtaining step and an estimating step. An active clay content estimation method in accordance with an aspect of the present invention includes an obtaining step and an estimating step.

In the active clay content estimation device and the active clay content estimation method, the obtaining step is a step, executed by the one or more processors, of obtaining information concerning mixed sand with which green sand containing clay is mixed. The estimating step is a step, executed by the one or more processors, of estimating an active clay content in the mixed sand in accordance with the information concerning the mixed sand.

A trained model generation device in accordance with an aspect of the present invention includes one or more processors configured to execute a constructing step. A trained model generation method in accordance with an aspect of the present invention includes a constructing step.

In the trained model generation device and the trained model generation method, the constructing step is a step of constructing, by machine learning, a trained model that takes, as an input, information concerning mixed sand with which green sand containing clay is mixed and outputs an active clay content in the mixed sand.

### Advantageous Effects of Invention

An aspect of the present invention makes it possible to estimate an active clay content in mixed sand.

### BRIEF DESCRIPTION OF DRAWINGS

FIG. 1 is a block diagram illustrating a configuration of an active clay content estimation system in accordance with Embodiment 1 of the present invention.

FIG. 2 is a block diagram illustrating a configuration of an information processing device in accordance with Embodiment 1 of the present invention.

FIG. 3 is a view schematically illustrating an example of a trained model in accordance with Embodiment 1 of the present invention.

FIG. 4 is a flowchart illustrating a flow of an active clay content estimation method in accordance with Embodiment 1 of the present invention.

FIG. 5 is a flowchart illustrating a flow of a trained model generation method in accordance with Embodiment 1 of the present invention.

FIG. 6 is a graph illustrating a relation between estimated active clay contents and actual active clay contents.

FIG. 7 is a graph illustrating a relation between estimated active clay contents and actual active clay contents.

### DESCRIPTION OF EMBODIMENTS

### Embodiment 1

### <Active Clay Content Estimation System>

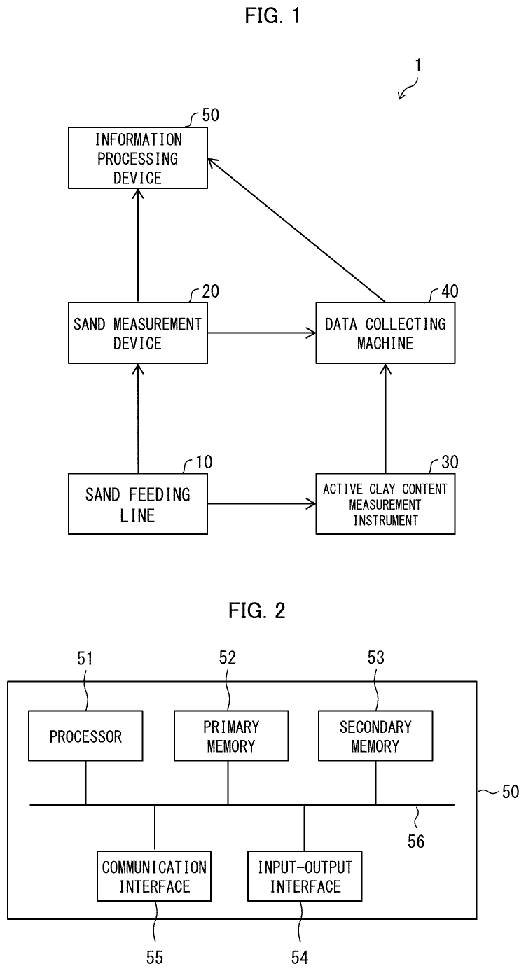

FIG. 1 is a block diagram illustrating a configuration of an active clay content estimation system 1 in accordance with Embodiment 1. The active clay content estimation system 1 is a system for estimating an active clay content in mixed sand. The mixed sand is sand which is used for molding and with which green sand containing clay is mixed. The green sand herein includes sand obtained as a result of shaking-out of a mold having been used in casting. The active clay content in the mixed sand refers to a percentage of clay retaining caking power, with respect to the clay included in the mixed sand.

The active clay content estimation system 1 executes two phases, i.e., an estimation phase and a training phase. The estimation phase is a phase in which the active clay content in the mixed sand is estimated in accordance with information concerning the mixed sand. The training phase is a phase in which a trained model used to estimate the active clay content in the mixed sand is constructed by machine learning in accordance with the information concerning the mixed sand.

The active clay content estimation system 1 includes a sand feeding line 10, a sand measurement device 20, an active clay content measurement instrument 30, a data collecting machine 40, and an information processing device 50.

In the estimation phase and training phase, the sand feeding line 10 feeds the mixed sand to the sand measurement device 20. In the training phase, a portion of the mixed sand in the sand feeding line 10 is taken as a sample, and an active clay content in the sample thus taken is measured by the active clay content measurement instrument 30.

The sand measurement device 20 is a device that measures information concerning the mixed sand fed by the sand feeding line 10. The sand measurement device 20 supplies, to the data collecting machine 40, the information concerning the mixed sand having been subjected to the measurement.

The information concerning the mixed sand is, for example, information indicating a property of the mixed sand. Examples of the information indicating the property of the mixed sand include a compressive strength, a shear strength, a moisture content, a permeability, compactability (CB value), and a sand temperature of the mixed sand. The compressive strength refers to a maximum stress with which a material can withstand, with respect to a compressive load. The shear strength refers to a maximum shearing stress with which a material can withstand without being broken. The moisture content of the mixed sand refers to, for example, a weight or a volume of moisture contained in unit weight or unit volume of the mixed sand. The permeability of the mixed sand refers to, for example, a degree of ease with which a gas (such as hydrogen, oxygen, nitrogen, carbon monoxide, carbon dioxide, and/or hydrocarbon) discharged from a mold passes through the mold. The compactability of the mixed sand refers to a value that indicates a moisture state in a particle surface layer of the mixed sand and that is used as an indicator of evaluation of the green sand. The sand temperature refers to a temperature of the mixed sand. Note that the information indicating the property of the mixed sand is not limited to these, and may include another kind of information.

Note that the examples of the information concerning the mixed sand include not only the information indicating the property of the mixed sand but also information concerning a composition of the mixed sand. Examples of the information concerning the composition of the mixed sand include an added amount of silica sand introduced into a sand mixer, an added amount of clay introduced into the sand mixer, and an added amount of an additive that is not the clay and that is introduced into the sand mixer. The clay introduced into the sand mixer is, for example, bentonite. The additive that is not the clay is, for example, an additive such as graphite, moisture, sea coal, or starch. The information concerning the composition of the mixed sand is not limited to these, and may include another kind of information. For example, the information concerning the composition of the mixed sand may include a content of the clay or a content of the additive. The information concerning the composition of the mixed sand may be a measured value or a set value. Measurement or setting of the information concerning the composition of the mixed sand is carried out by sand processing equipment (for example, a sand mixer).

The information concerning the mixed sand is not limited to those described above, and may include another kind of information. The information concerning the mixed sand may include, for example, a mixing period of the sand mixer. Measurement or setting of the mixing period is carried out by, for example, the sand processing equipment, similarly to the measurement or setting of the information concerning the composition of the mixed sand.

The active clay content measurement instrument 30 is an instrument that measures an active clay content in the mixed sand. The active clay content measurement instrument 30 measures the active clay content by a spotting technique, for example. According to the spotting method, first, a given amount of the mixed sand is weighed, and is dispersed, as a dispersant, in water with use of sodium pyrophosphate, whereby a suspension is prepared. Then, a methylene blue solution, which is used as a coloring agent, is added to the suspension one or more times until a light-blue halo is observed around a dark-blue spot. In doing so, 0.5 to 1.0 (ml) of the methylene blue solution is added each time. The halo is observed to measure an active clay content. Note that the method for measuring the active clay content is not limited to the spotting technique, and may be another method.

In the estimation phase, the data collecting machine 40 collects information concerning the mixed sand from the sand measurement device 20. In the training phase, the data collecting machine 40 collects the information concerning the mixed sand from the sand measurement device 20 and the active clay content from the active clay content measurement instrument 30, thereby collecting a set of the information concerning the mixed sand and the active clay content as training data. In a case where the information concerning the composition of the mixed sand is used as the information concerning the mixed sand, the data collecting machine 40 collects, in the estimation phase and learning phase, the information concerning the composition of the mixed sand from the sand processing equipment. Also in a case where the mixing period is used as the information concerning the mixed sand, a similar process to that in the case of using the information concerning the composition of the mixed sand is carried out.

The information processing device 50 is one example of the active clay content estimation device herein, and estimates the active clay content in the mixed sand in accordance with the information concerning the mixed sand. Also, the information processing device 50 is one example of the trained model generation device herein, and constructs, by machine learning, a trained model used to estimate the active clay content in the mixed sand in accordance with the information concerning the mixed sand.

FIG. 2 is a block diagram illustrating a configuration of the information processing device 50. The information processing device 50 is achieved by a general-purpose computer, and includes a processor 51, a primary memory 52, a secondary memory 53, an input-output interface 54, a communication interface 55, and a bus 56. The processor 51, the primary memory 52, the secondary memory 53, the input-output interface 54, and the communication interface 55 are connected to each other via the bus 56.

The secondary memory 53 has an active clay content estimation program P1, a trained model generation program P2, and a trained model LM1 stored therein. The processor 51 reads the active clay content estimation program P1 stored in the secondary memory 53 so as to load the active clay content estimation program P1 into the primary memory 52, and executes each step included in an active clay content estimation method M1 in accordance with a command included in the active clay content estimation program P1 loaded in the primary memory 52.

The processor 51 reads the trained model generation program P2 stored in the secondary memory 53 so as to load the trained model generation program P2 into the primary memory 52, and executes each step included in a trained model generation method M2 in accordance with a command included in the trained model generation program P2 loaded in the primary memory 52. Note that the phrase “the secondary memory 53 has the active clay content estimation program P1 and the trained model generation program P2 stored therein” means that a source code or an executable file obtained by compiling the source code is stored in the secondary memory 53.

The processor 51 reads the trained model LM1 stored in the secondary memory 53 so as to load the trained model LM1 into the primary memory 52. The trained model LM1 loaded in the primary memory 52 is used by the processor 51 to execute the active clay content estimation method M1. Note that the phrase “the secondary memory 53 has the trained model LM1 stored therein” means that a parameter defining the trained model LM1 is stored in the secondary memory 53.

A device that can be used as the processor 51 is, for example, a central processing unit (CPU). A device that can be used as the primary memory 52 is, for example, a random access memory (RAM). A device that can be used as the secondary memory 53 is, for example, a flash memory.

The input-output interface 54 is connected to an input device and/or an output device. The input-output interface 54 is, for example, USB. Data obtained in the active clay content estimation method M1 or the trained model generation method M2 is input to the information processing device 50 via the input-output interface 54. Information to be supplied to a user in the active clay content estimation method M1 or the trained model generation method M2 is output from the information processing device 50 via the input-output interface 54.

The communication interface 55 is an interface used to carry out communication with another computer. The communication interface 55 can include an interface used to carry out communication with another computer not via a network, for example, a Bluetooth (registered trademark) interface. The communication interface 55 can also include an interface used to carry out communication with another computer via local area network (LAN), for example, a Wi-Fi (registered trademark) interface.

Embodiment 1 employs a configuration that uses a single processor (processor 51) to execute the active clay content estimation method M1 and the trained model generation method M2. However, the present invention is not limited to this configuration. That is, a configuration that uses a plurality of processors to execute the active clay content estimation method M1 and the trained model generation method M2 may alternatively be employed. In this case, the plurality of processors that work together to execute the active clay content estimation method M1 and the trained model generation method M2 may be provided in a single computer such that the plurality of processors can communicate with each other via a bus. Alternatively, the plurality of processors may be separately provided in a plurality of computers such that the plurality of processors can communicate with each other via a network.

For example, a computer that executes the active clay content estimation method M1 and a computer that executes the trained model generation method M2 may be provided as separate devices. For example, the following aspect is possible. That is, a processor integrated in a computer constituting a cloud server and a processor integrated in a computer owned by a user of the cloud server work together to execute the active clay content estimation method M1 and the trained model generation method M2.

Embodiment 1 employs a configuration in which the trained model LM1 is stored in the memory (secondary memory 53) integrated in the computer including the processor (processor 51) that executes the active clay content estimation method M1 and the trained model generation method M2. However, the present invention is not limited to this configuration. That is, the following configuration may alternatively be employed: the trained model LM1 is stored in a memory integrated in a computer that is not the computer including the processor that executes the active clay content estimation method M1 and the trained model generation method M2. In this case, the computer containing the memory having the trained model LM1 stored therein is configured to be communicable, via a network, with the computer including the processor that executes the active clay content estimation method M1 and the trained model generation method M2. For example, the following configuration can be employed: the trained model LM1 is stored in a memory contained in a computer constituting a cloud server; and a processor contained in a computer owned by a user of the cloud server executes the active clay content estimation method M1 and the trained model generation method M2.

Embodiment 1 employs a configuration in which the trained model LM1 is stored in a single memory (secondary memory 53). However, the present invention is not limited to this configuration. That is, the following configuration may alternatively be employed: the trained model LM1 is divided into a plurality of parts and stored in a respective plurality of memories. In this case, the plurality of memories in which the parts of the trained model LM1 are stored may be provided in a single computer (which may be or may not be the computer containing the processor that executes the active clay content estimation method M1 and the trained model generation method M2) or in a respective plurality of different computers (which may or may not include the computer containing the processor that executes the active clay content estimation method M1 or the trained model generation method M2). For example, the following configuration may be employed: the trained model LM1 is divided into a plurality of parts and stored in a respective plurality of memories contained in a respective plurality of computers constituting a cloud server.

The trained model LM1 is a trained model used to estimate the active clay content in the mixed sand. The trained model LM1 is a model constructed by machine learning so as to take, as an input, the information concerning the mixed sand and to output the active clay content in the mixed sand. Examples of the trained model LM1 include algorithms such as neural network models (e.g., convolutional neural network and recurrent neural network), regression models (e.g., linear regression and nonlinear regression), and tree models (e.g., regression tree). Note that, in Embodiment 1, the compressive strength in the information concerning the mixed sand is employed as an input to the trained model LM1.

The active clay content is an indicator for measuring an amount of effectively-functioning clay in the mixed sand. Increase in the effective clay content in the mixed sand results in stronger bonding between sand particles, thereby increasing the compressive strength. That is, the compressive strength and the active clay content are correlated to each other. Thus, employing the compressive strength as an input to the trained model LM1 makes it possible to accurately estimate the active clay content.

Further, the shear strength and the compressive strength are strongly correlated to each other. Thus, increase in the effective clay content in the mixed sand results in stronger bonding between sand particles, thereby increasing the compressive strength. Consequently, the shear strength is increased. That is, the shear strength and the active clay content are correlated with each other.

Therefore, employing, instead of or together with the compressive strength, the shear strength as an input to the trained model LM1 also makes it possible to accurately estimate the active clay content.

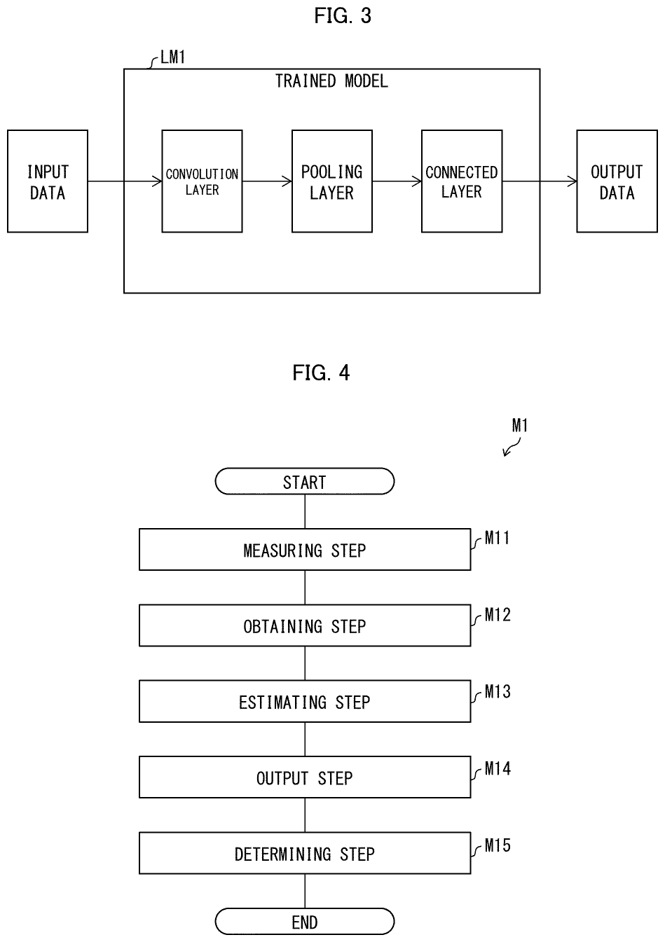

FIG. 3 is a view schematically illustrating an example of the trained model LM1 in accordance with Embodiment 1. FIG. 3 illustrates, as an example, a case in which the trained model LM1 is convolutional neural network (CNN). In FIG. 3, input data is input to the trained model LM1. The trained model LM1 is made of, for example, a convolution layer, a pooling layer, and a connected layer. In the convolution layer, the input data is filtered so that information is convoluted. The data having undergone the convolution is subjected to a pooling process in the pooling layer. This can enhance the recognition capability of the model with respect to a positional change of a feature in the data. The data having undergone the pooling process is processed in the connected layer, so as to be converted into output data of the trained model LM1, i.e., a label for specifying the active clay content. Then, the output data is output.

That is, input data input to the trained model LM1 is caused to pass through the layers shown in FIG. 3 in the illustrated order, and consequently an estimation result of the active clay content is output. Note that an output form of the estimation result is not particularly limited. For example, the active clay content may be indicated in the form of text data.

### <Flow of Active Clay Content Estimation Method>

The following description will discuss, with reference to FIG. 4, a flow of the active clay content estimation method M1 in accordance with Embodiment 1. FIG. 4 is a flowchart illustrating a flow of the active clay content estimation method M1. The active clay content estimation method M1 includes a measuring step M11, an obtaining step M12, an estimating step M13, an output step M14, and a determining step M15.

In the measuring step M11, the sand measurement device 20 measures the information concerning the mixed sand fed by the sand feeding line 10. The sand measurement device 20 supplies the information concerning the mixed sand to the information processing device 50 (in Embodiment 1, the information concerning the property of the mixed sand, more specifically, the compressive strength of the mixed sand). The obtaining step M12 is a step in which the processor 51 obtains the information concerning the mixed sand from the sand measurement device 20. In a case where the measurement value concerning the composition of the mixed sand is used as the information concerning the mixed sand, the sand processing equipment measures the information concerning the composition of the mixed sand in the measuring step M1. Meanwhile, in a case where the setting value concerning the composition of the mixed sand is used as the information concerning the mixed sand, the measuring step M11 is omitted. In each of these cases, in the obtaining step M12, the processor 51 obtains the information concerning the composition of the mixed sand from the sand processing equipment. Also in a case where the measurement value or the setting value of the mixing period is used as the information concerning the mixed sand, a similar process to that in the case of using the measurement value or the setting value of the composition of the mixed sand is carried out.

The estimating step M13 is a step in which the processor 51 estimates, in accordance with the information concerning the mixed sand obtained in the obtaining step M12, an active clay content in the mixed sand. For example, the processor 51 estimates the active clay content in the mixed sand with use of the trained model LM1 that is constructed by machine learning and that takes, as an input, the information concerning the mixed sand and outputs the active clay content.

For example, the processor 51 may output the active clay content estimated in the estimating step M13 to a display device connected to the input-output interface 54 or the communication interface 55. From a content displayed by the display device, a user of the information processing device 50 can acknowledge the active clay content in the mixed sand.

The output step M14 is a step in which the processor 51 outputs an alert in a case where the active clay content estimated in the estimating step M13 satisfies a given condition. For example, the processor 51 may output a message to the display device connected to the input-output interface 54 or the communication interface 55. For another example, the processor 51 may output a warning sound or an audio message to a speaker connected to the input-output interface 54 or the communication interface 55. For example, the processor 51 outputs an alert in a case where the active clay content is too high or too low (i.e., in a case where the active clay content is outside a range defined by a threshold). Alternatively, for example, the processor 51 may output an alert in a case where the estimated active clay content is within the range defined by the threshold. The processor 51 may change the output mode of the alert in accordance with the active clay content. For example, in a configuration in which the alert is output by the speaker, the processor 51 may change a sound volume in accordance with a degree of excess and deficiency of the active clay content. For another example, the processor 51 may carry out a control in the following manner. That is, in a case where the active clay content satisfies a first condition, an alert is given via display output. Meanwhile, in a case where the active clay content satisfies a second condition, an alert is given via display output and audio output.

The determining step M15 is a step in which the processor 51 determines, in accordance with the active clay content estimated in the estimating step M13, an added amount of the clay to be introduced into the sand mixer. For example, the processor 51 may determine the added amount of the clay so that the added amount is reduced as the active clay content estimated in the estimating step M13 increases. For example, the processor 51 may output the determined added amount to the output device (e.g., the display device, speaker) connected to the input-output interface 54 or the communication interface 55. For another example, the processor 51 may transmit, to the sand mixer, control information corresponding to the determined added amount and may control the sand mixer so that the determined added amount of the clay is introduced into the sand mixer.

The processor 51 may determine a mixing condition of the sand mixer in accordance with the estimated active clay content. The mixing condition may be, for example, a rotation speed (in a case of continuous process and batch process) and a rotation period (in a case of batch process) of a mixing section. The mixing section is a member for mixing the mixed sand. For example, the mixing section is, for example, a muller tire, a muller wheel, or a propeller-type or screw-type mixing blade. The rotation speed of the mixing section refers to, for example, the number of rotations of the mixing section per unit time. The rotation period of the mixing section refers to, for example, a period of time in which the mixing section rotates in a single process. The processor 51 may output the determined mixing condition to the output device. Alternatively, the processor 51 may supply, to the sand mixer, a control signal generated in accordance with the determined mixing condition.

<Flow of trained model generation method>

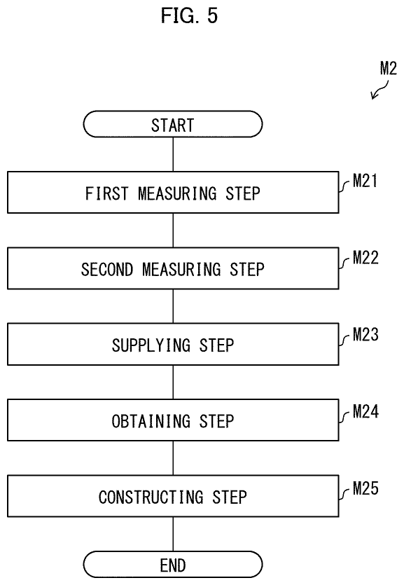

The following description will discuss, with reference to FIG. 5, a flow of the trained model generation method M2 in accordance with Embodiment 1. FIG. 5 is a flowchart illustrating a flow of the trained model generation method M2. The trained model generation method M2 includes a first measuring step M21, a second measuring step M22, a supplying step M23, an obtaining step M24, and a constructing step M25. Note that a part of the steps may be executed in parallel or in a different order.

In the first measuring step M21, the sand measurement device 20 measures information concerning mixed sand with use of the mixed sand fed by the sand feeding line 10. The sand measurement device 20 supplies the information concerning the mixed sand to the data collecting machine 40.

In the second measuring step M22, the active clay content measurement instrument 30 measures an active clay content in the mixed sand in the sand feeding line 10.

The active clay content measurement instrument 30 measures the active clay content by the spotting method, for example. Note that the method for measuring the active clay content is not limited to the spotting technique, and may be another method. The active clay content measurement instrument 30 supplies, to the data collecting machine 40, information indicating the measured active clay content.

In the supplying step M23, the data collecting machine 40 supplies, to the information processing device 50, training data (dataset-for-learning) including a combination of (i) the information concerning the mixed sand obtained from the sand measurement device 20 and (ii) a measured value of the active clay content in the mixed sand. The obtaining step M24 is a step in which the processor 51 obtains the training data from the data collecting machine 40. The information concerning the mixed sand (in Embodiment 1, the information concerning the property of the mixed sand, more specifically, the compressive strength of the mixed sand) is a measurement value obtained as a result of the measurement carried out by the sand measurement device 20. The measured value of the active clay content in the mixed sand is a value indicating a result of the measurement carried out by the active clay content measurement instrument 30. In a case where the information concerning the composition of the mixed sand is used as the information concerning the mixed sand, the information concerning the mixed sand is a setting value set by the sand processing equipment or a measurement value measured by the sand processing equipment. Also in a case where the mixing period is used as the information concerning the mixed sand, a similar process to that in the case of using the information concerning the composition of the mixed sand is carried out.

The constructing step M25 is a step in which the processor 51 constructs, by machine learning, the trained model LM1 that takes, as an input, the information concerning the mixed sand and outputs the active clay content in the mixed sand. For example, the processor 51 constructs the trained model LM1 by a learning technique of convolutional neural network.

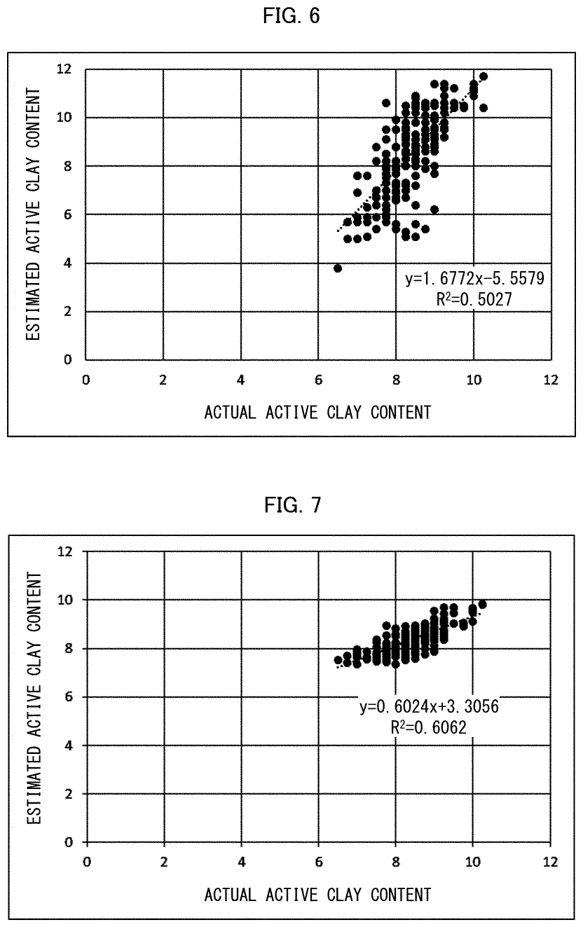

FIG. 6 is a graph illustrating a relation between (i) active clay contents estimated by the processor 51 in a case of employing a compressive strength as an input and (ii) actual active clay contents. In the example shown in FIG. 6, the coefficient of determination is 0.5027. This shows that the active clay content can be accurately estimated on the basis of the compressive strength. The same as the compressive strength is also true for the shear strength, which exhibits a strong correlation with the compressive strength.

### Variation 1

The description in Embodiment 1 has dealt with the configuration in which the compressive strength of the mixed sand is employed as an input to the trained model LM1. However, the present invention is not limited to the configuration in which only the compressive strength of the mixed sand is employed as an input to the trained model LM1. For example, in an alternative configuration, at least one parameter selected from the group consisting of a moisture content, a permeability, compactability, and a sand temperature may be employed as an input to the trained model LM1, in addition to the compressive strength of the mixed sand.

The compressive strength is affected not only by the active clay content but also by the moisture content, the permeability, the compactability, the sand temperature, and the like. For example, even if the active clay content is constant, a change in the moisture content will change the compressive strength. Particularly, in a case where the moisture content is too large, the compressive strength tends to decrease. Thus, by additionally employing these parameters, which affect the compressive strength, as inputs to the trained model LM1, the accuracy in estimating the active clay content is enhanced. Note that the same is also true in a case where the shear strength of the mixed sand is employed as an input to the trained model LM1 in addition to or instead of the compressive strength of the mixed sand.

FIG. 7 is a graph illustrating a relation between (i) active clay contents estimated by the processor 51 in a case of employing, as inputs, a compressive strength, compactability, a sand temperature, and a permeability and (ii) actual active clay contents. In the example shown in FIG. 7, the coefficient of determination is 0.6062. This shows that the active clay content can be estimated more accurately than in the case of employing only the compressive strength as an input (see FIG. 6). The same as the compressive strength applies is also true for the shear strength, which exhibits a strongly correlation with the compressive strength.

### Variation 2

The description in Embodiment 1 has dealt with the configuration in which the compressive strength of the mixed sand is employed as an input to the trained model LM1. However, the present invention is not limited to the configuration in which only the compressive strength of the mixed sand is employed as an input to the trained model LM1. For example, in an alternative configuration, at least one parameter selected from the group consisting of an added amount of silica sand introduced into a sand mixer, an added amount of clay introduced into the sand mixer, and an added amount of an additive that is not the clay and that is introduced into the sand mixer may be employed as an input to the trained model LM1, in addition to the compressive strength of the mixed sand.

The compressive strength is affected not only by the active clay content but also by an amount of fine particles contained in the sand, a particle size of the sand, and/or the like. These values change depending on the added amount of the silica sand, the added amount of the clay, and the added amount of the additive that is not the clay. Thus, by additionally employing these parameters, which affect the compressive strength, as inputs to the trained model LM1, the accuracy in estimating the active clay content is enhanced. Note that the same applies also in a case where the shear strength of the mixed sand is employed as an input to the trained model LM1 in addition to or instead of the compressive strength of the mixed sand.

### Effects of Embodiment 1

As discussed above, in accordance with Embodiment 1, the information processing device 50 estimates an active clay content in mixed sand in accordance with information concerning the mixed sand with which green sand containing clay is mixed. Thus, with the active clay content estimation system 1, it is not necessary to measure, by the spotting technique or the like, the active clay content in the mixed sand in a sand treatment cycle. This eliminates the need for the active clay content measurement instrument 30 in the sand treatment cycle. With this, in accordance with Embodiment 1, it is possible to estimate the active clay content in the mixed sand without decreasing the processing speed of the entire sand treatment cycle. That is, in accordance with Embodiment 1, the information processing device 50 can estimate the active clay content in the mixed sand with the sand treatment cycle not including the measurement of the active clay content in the mixed sand.

In accordance with Embodiment 1, the information processing device 50 can estimate the active clay content in the mixed sand with use of either or both of a compressive strength and a shear strength of the mixed sand.

Further, in accordance with Embodiment 1, the information processing device 50 can estimate the active clay content in the mixed sand with use of at least one parameter selected from the group consisting of a moisture content, a permeability, compactability, and a sand temperature of the mixed sand. In accordance with Embodiment 1, the information processing device 50 can estimate the active clay content in the mixed sand with use of at least one parameter selected from the group consisting of an added amount of silica sand introduced into the sand mixer, an added amount of clay introduced into the sand mixer, and an added amount of an additive that is not the clay and that is introduced into the sand mixer.

In accordance with Embodiment 1, the information processing device 50 can estimate the active clay content in the mixed sand by inputting the information concerning the mixed sand into the trained model LM1 constructed by machine learning.

The information processing device 50 in accordance with Embodiment 1 outputs an alert in a case where the estimated active clay content satisfies a give condition. With this, for example, in a case where the active clay content is too high or too low, the user of the information processing device 50 can acknowledge that. The information processing device 50 in accordance with Embodiment 1 determines an added amount of the clay in accordance with the estimated active clay content. This makes it possible to introduce an appropriate amount of the clay into the sand mixer.

In accordance with Embodiment 1, use of the constructed trained model LM1 makes it possible to estimate the active clay content in the mixed sand with the sand treatment cycle not including the measurement of the active clay content in the mixed sand.

### Supplementary Remark 1

In accordance with the above-described embodiment, the information processing device 50 estimates the active clay content in the mixed sand with use of the trained model LM1 constructed by machine learning. The method for estimating the active clay content in the mixed sand is not limited to a method involving use of the trained model LM1, and may be another method. For example, the information processing device 50 may estimate the active clay content in the mixed sand with use of a table in which the information concerning the mixed sand and the active clay content in the mixed sand are stored in association with each other. In this case, for example, the information processing device 50 obtains the information concerning the mixed sand from the sand measurement device 20, searches the table for the obtained information, and reads out, from the table, the active clay content associated with the searched information. In this manner, the information processing device 50 specifies the active clay content.

### Supplementary Remark 2

The above-described embodiment has exemplified a case where the convolutional neural network is employed as the trained model LM1 used to estimate the active clay content. The trained model LM1 is not limited to the convolutional neural network. The trained model LM1 may be, for example, a nonlinear relational formula expressing a relation between an explanatory variable and an objective variable which nonlinear relational formula is specified with use of a nonlinear regression algorithm. In other words, the processor 51 may use the nonlinear regression algorithm to specify the nonlinear relational formula expressing the relation between the explanatory variable and the objective variable.

The nonlinear regression algorithm may be, for example, a genetic algorithm or a Monte Carlo method.

According to the genetic algorithm, a plurality of individuals, which are candidate solutions represented as genes, are prepared. Then, among these, individuals having a higher fitness are preferentially selected and are subjected to operations such as crossover and mutation. Through repetition of this operation, a solution is found. In this case, each individual represents a nonlinear relational formula in a tree structure, for example. An operator and an argument included in the relational formula are represented as nodes of the tree. The fitness is given by a fitness function.

In a case where the Monte Carlo method is employed, the information processing device 50 operates as follows, for example. That is, the information processing device 50 randomly selects a length of a relational formula and elements (e.g., a variable and an operator) of the relational formula, so as to generate a plurality of relational formulae. Then, among the plurality of relational formulae thus generated, the information processing device 50 selects a relational formula having a smallest error. In this manner, a relational formula representing a relation between an explanatory variable and an objective variable is constructed.

### Supplementary Remark 3

In a case where the trained model LM1 is a nonlinear relational formula representing a relation between an explanatory variable and an objective variable, the processor 51 may generate, by a second trained model, the nonlinear relational formula (trained model LM1) representing the relation between the explanatory variable and the objective variable. The second trained model is a trained model that is constructed by machine learning and that takes, as an input, information concerning mixed sand and an active clay content in the mixed sand and outputs a nonlinear relational formula. For example, the second trained model may be a model constructed by a learning method of recurrent neural network (RNN).

### Supplementary Remark 4

The description of the above-described embodiment has dealt with a case in which the information processing device 50 estimates a single active clay content with use of a single trained model LM1. The method for estimating the active clay content is not limited to those indicated in the above-described embodiment. For example, the information processing device 50 may estimate a plurality of active clay contents with use of a plurality of trained models. In this case, for example, the information processing device 50 may determine, as a final estimated active clay content, an average of a plurality of estimated active clay contents obtained by estimation involving use of the plurality of trained models.

The average of the plurality of estimated active clay contents may be a weighted average. In this case, the information processing device 50 may change, in accordance with the property or composition of the mixed sand, a weight to be assigned in obtaining of a weighted average. For example, in a case where compactability is not less than a given threshold, the information processing device 50 may carry out control to increase the weight of, among the plurality of estimated active clay contents, an active clay content obtained by estimation involving use of a given trained model among the plurality of trained models.

### Supplementary Remark 5

The present invention is not limited to the embodiments described herein, but can be altered by a skilled person in the art within the scope of the claims. The present invention also encompasses, in its technical scope, any embodiment derived by combining technical means disclosed in differing embodiments.

### REFERENCE SIGNS LIST

50: information processing device
51: processor
LM1: trained model
M1: active clay content estimation method
M2: trained model generation method

1. An active clay content estimation device comprising:
one or more processors,
the one or more processors being configured to execute:
an obtaining step of obtaining information concerning mixed sand with which green sand containing clay is mixed; and
an estimating step of estimating an active clay content in the mixed sand in accordance with the information concerning the mixed sand.

2. The active clay content estimation device as set forth in claim 1, wherein the information concerning the mixed sand includes either or both of a compressive strength and a shear strength of the mixed sand.

3. The active clay content estimation device as set forth in claim 2, wherein the information concerning the mixed sand further includes at least one parameter selected from the group consisting of a moisture content, a permeability, compactability, and a sand temperature of the mixed sand.

4. The active clay content estimation device as set forth in claim 2, wherein the information concerning the mixed sand includes at least one at least one parameter selected from the group consisting of an added amount of silica sand, an added amount of the clay, and an added amount of an additive that is not the clay.

5. The active clay content estimation device as set forth in claim 1, wherein
in the estimating step, the one or more processors estimate the active clay content in the mixed sand with use of a trained model that is constructed by machine learning and that takes, as an input, the information concerning the mixed sand and outputs the active clay content.

6. The active clay content estimation device as set forth in claim 1, wherein
the one or more processors are configured to further execute:
an output step of outputting an alert in a case where the active clay content estimated in the estimating step satisfies a given condition.

7. The active clay content estimation device as set forth in claim 1, wherein
the one or more processors are configured to further execute:
a determining step of determining an added amount of the clay in accordance with the active clay content estimated in the estimating step.

8. A trained model generation device comprising:
one or more processors,
the one or more processors being configured to execute:
a constructing step of constructing, by machine learning, a trained model that takes, as an input, information concerning mixed sand with which green sand containing clay is mixed and outputs an active clay content in the mixed sand.

9. An active clay content estimation method comprising the steps executed by one or more processors, the steps being:
an obtaining step of obtaining information concerning mixed sand with which green sand containing clay is mixed; and
an estimating step of estimating an active clay content in the mixed sand in accordance with the information concerning the mixed sand.

10. (canceled)
